(19) United States
(12) Patent Application Publication
(10) Pub. No.: US 2004/0126896 A1
(43) Pub. Date: Jul. 1, 2004

(54) Method for determining multimers of plasma proteins

(76) Inventors: Peter Hermentin, Marburg (DE); Thomas Cuesta-Linker, Wetter (DE); Karl-Heinz Schmidt, Weimar (DE)

Correspondence Address: Finnegan, Henderson, Farabow,, Garrett & Dunner, L.L.P., 1300 I Street, N.W., Washington, DC, 20005-3315, US

(21) Appl. No.: 10/682,199

(22) Filed: Oct. 10, 2003

(30) Foreign Application Priority Data
Oct. 14, 2002 (DE) 10247876.7

(51) Int. Cl. G01N 33/53; G01N 33/542; G01N 33/557

(52) U.S. Cl. 436/517

(57) ABSTRACT

A method for the qualitative and quantitative determination of multimers of fibrinogen and of von Willebrand factor by gel electrophoresis, in which a sample containing von Willebrand factor (vWF) or fibrinogen is fractionated by submarine electrophoresis using a continuous, homogeneous agarose gel free of lumps, and the multimer bands are visualized immunochemically after a western blot analysis by a specific antibody-enzyme conjugate on the blotting membrane or by a suitable dye, preferably with a blue stain, in the gel, is described.

The invention relates to a method for the qualitative and quantitative determination of the multimers of plasma proteins by gel electrophoresis.

It is known that constituents of the plasma which are important for coagulation occur not only in monomeric form but also as multimeric molecules in the plasma. In some cases, a different therapeutic value can be attributed to the individual monomers. Examples of such multimer-forming therapeutic proteins are fibrinogen and von Willebrand factor. Quantitative and qualitative determination of the multimers is of considerable importance for diagnosing the cause of a coagulation defect on the one hand, but also for identifying the quality of a coagulation product on the other hand. Determination of fibrinogen and of von Willebrand factor multimers therefore deserves particular attention.

Fibrinogen has a quite essential function in hemostasis. It is converted through the action of thrombin to fibrin which crosslinks to give fine fibrin fibers which are stabilized by factor XIII. A blood clot which ensures hemostasis is formed therefrom. The integrity and the structure of fibrinogen is of great importance for hemostatic effect.

Although fibrinogen itself is not prone to polymerization, there is also under suitable conditions, such as high concentration, low ionic strength and long incubation at 4° C., also the formation of fibrinogen multimers similar to fibrin. Fibrinogens of this type can be analyzed and visualized by SDS agarose gel electrophoresis with Western blotting and immunochemical staining, as described in the international patent application WO 01/12244.

The methods for separating fibrinogen multimers by SDS agarose gel electrophoresis were introduced by Connaghan et al. (1) and Proietti et al. (2). These methods were then improved by the method described in the international patent application WO 01/12244, in which the method described by Raines et al. (3) for separating von Willebrand multimers was modified and utilized for fibrinogen electrophoresis. This entailed the horizontal electrophoresis being carried out essentially as described in (3) and the detection of fibrinogen multimers being carried out essentially as described in (2).

However, the electrophoresis method of WO 01/12244 is rather complicated and time-consuming and requires very specific experience. Moreover, three different gels are required, namely a stacking gel (1% agarose dissolved in 70 mM tris, 4 mM EDTA, 4% SDS), a cathode gel (1.6% agarose dissolved in 100 mM tris, 150 mM glycine, 0.1% SDS) and a separating gel (2% agarose dissolved in 200 mM tris, 100 mM glycine, 0.4% SDS) which are cast at a temperature above 50° C. and must be left to stand for at least 2 hours for the polymerization. Samples are loaded at 0.6 to 0.9 μl per lane. The electrophoresis is carried out at 600 to 650V and at a temperature of 16° C. in a flat bed apparatus. The running buffer is connected to the gel over a length of 4 to 8 filter papers immersed in the electrode buffer.

The preparation of three different agarose gels requires considerable experience because the process of swelling the agarose must be carried out at near the boiling point of water. It is moreover essential to find the optimal time at which, on the one hand, the agarose has dissolved sufficiently for lumps (solid residues) to be no longer present and, on the other hand, no agarose has yet been redeposited as precipitate on the upper, cold rim of the glass. Casting the gel also requires special skill, in particular introduction of the loading wells. The removal of the comb necessary for forming the loading wells from the cooled, hardened gel may easily lead to the agarose gel being damaged and thus the cast gel being unusable.

Similar difficulties also occur in the methods customary to date for fractionating von Willebrand factor (vWF) multimers.

Functional von Willebrand factor (vWF), a glycoprotein, circulates in the bloodstream with a diverse molecular weight distribution, called multimers, and the multimers may have a molecular weight distribution from 500 kD up to 20,000 kD. The smallest unit thereof is the dimer with a molecular weight of about 550 kD; it consists of two monomers which are connected together by disulfide bridges. Polymers, called multimers, with a molecular weight of up to 20,000 kD, are produced from these dimers by further disulfide linkages.

The clinical manifestation of von Willebrand disease is very heterogeneous and extends from mild forms such as type 1 up to very severe deficiency states with spontaneously occurring hemorrhages in type 3. The proneness to bleeding is caused by quantitative and/or qualitative impairments of the two main functions of vWF: mediating the adhesion of platelets with one another and to the injured vessel wall, and stabilization of the coagulation factor VIII. It is thus possible for both the early primary and the secondary, later onset, hemostasis to be impaired.

In a secondary coagulation defect caused by a factor VIII deficiency, however, factor VIII is not inevitably reduced; on the contrary, the coagulation defect may also be the expression of a quantitative and/or qualitative defect in von Willebrand factor.

Owing to the complexity of the coagulation defect, the diagnosis and classification of von Willebrand disease into its various degrees of severity is a challenge to every coagulation laboratory. Accurate diagnosis, i.e. determination of the subtype, has important consequences for the patient himself, since only then is it possible to select an optimal treatment and, in the case of a generic defect to provide more detailed advice. Thus, treatment with desmopressin, which is outstandingly effective for type 1 diseases, may be inadequate or even contraindicated for severe von Willebrand diseases of type 3 and many of the type 2 forms, so that a vWF-containing factor VIII concentrate must be given in these cases. Good therapeutic efficacy is shown in these cases in particular by factor VIII concentrates with large vWF multimers.

vWF is not a plasma coagulation protein (like factor VIII or IX), but acts by forming bridges between the injured vessel wall and the platelets as an important constituent of primary hemostasis and as a so-called adhesive for the platelets. In addition, it stabilizes factor VIII, which is able to circulate in the blood for only a very short time without binding to vWF because, otherwise, it is rapidly degraded.

Information is obtained according to the invention about structural changes in the vWF molecule by visualizing the multimers by means of an SDS agarose gel electrophoresis with Western blot immunostain analysis (SAGE-WISA) or by Coomassie blue staining of the gel (SAGE-CB). The electrophoretic visualization of the multimers then permits conclusions to be drawn about structural peculiarities. Despite the possibility of detecting with great sensitivity vWF molecules from which the large multimers are absent using a collagen binding assay, multimer analysis is an important diagnostic test for patients with hereditary but also in particular acquired changes in vWF. Establishment of the multimer composition is also of great importance for assessing factor VIII coagulation products.

vWF multimer analysis by SAGE-WISA is state of the art and has been described by Metzner et al. (4). However, the method described therein is complicated and time-consuming. In addition, it suffers from the lack of reproducibility, making reliable quantification of the multimer bands difficult. Once again there are also the difficulties of casting a homogeneous agarose gel free of lumps, and the need to use several different agarose gels. Both in the method of (4) and in comparable methods (5,6,7) there is always the need for a separating gel and a stacking gel different therefrom. For example (4) uses a 0.9% agarose separating gel and a 0.8% agarose stacking gel, while (5) employs a 2% agarose separating gel and a 1% agarose stacking gel.

In addition, different agarose types are specified for the stacking gel and the separating gel. Thus, (4) specifies an HGT (high gelling temperature) agarose for the stacking gel and an LGT (low gelling temperature) agarose for the separating gel, while other agarose types are employed in other known separation methods. An additional difficulty which often arises with conventional gel electrophoretic separation methods is that the agarose gel shows a tendency to float. In this case, the agarose gel loses its adhesion to the bottom of the electrophoresis chamber, resulting in the formation of slanted, distorted lane patterns or even electrophoretic migration of proteins out of the gel and thus loss of protein bands. Although it is possible to prevent floating of the agarose gel by a catamaran-like weighting of the gel, this compresses the gel, which in turn either impairs band separation or reduces the number of lanes which can be used on each gel.

The aforementioned difficulties can now be solved by a method for the qualitative and quantitative determination of the multimers of fibrinogen or of von Willebrand factor by gel electrophoresis when a sample containing von Willebrand factor (vWF) or fibrinogen is fractionated by submarine electrophoresis using a continuous, homogeneous agarose gel free of lumps while cooling to 8° C. to 12° C., and the multimer bands are visualized immunochemically after a Western blot analysis by a specific antibody-enzyme conjugate on the blotting membrane or by a suitable dye, preferably with a blue stain, in the gel.

Although self-preparation of the homogeneous agarose gels free of lumps to be employed according to the invention is possible with sufficient experience, they are generally purchased, for example from Elchrom or Bio-Rad. Agarose gels of this kind, which have to date been employed almost exclusively for fractionating DNA or RNA samples, have only a separating gel with a uniform agarose concentration and do not require a separate stacking gel. The separation in this case is carried out in the following way:

A. Fractionation of vWF Multimers

The agarose gel employed for separating the vWF multimers had an agarose concentration of from 0.7 to 1.8 percent by weight, preferably from 0.8 to 1.2 percent by weight. An agarose gel without backing (e.g. from Bio-Rad) proved to be particularly advantageous for subsequent Western blot immunostain analysis (WISA). An agarose gel which proved to be particularly advantageous for Coomassie blue staining was one applied to a backing sheet (e.g. from Elchrom Scientific AG) because this allowed the agarose to be dried better at elevated temperature, preferably between 30° C. and 50° C., and more rapidly stained with Coomassie blue, the dye particularly preferred for this purpose.

The submarine electrophoresis took place with cooling to 10° C. in an SEA 2,000 HIPER electrophoresis chamber (Elchrom Scientific, Cham, Switzerland) with 0.8-1.5% agarose gels and, for example, an electrophoresis buffer composed of tris-glycine buffer with 0.1% SDS (mixture: 12.12 g of tris, 57.8 g of glycine and 2 g of SDS were made up to 2,000 ml with deionized water). The electrophoresis chamber was charged with 2,000 ml of electrophoresis buffer. The gel was then inserted and re-equilibrated in a preliminary electrophoresis. The preliminary electrophoresis took place at 25 V/max. watt/0.5 h (with circulating pump running in order to make rapid re-equilibration of the gel possible).

For the electrophoresis, the gel was placed in the electrophoresis chamber in such a way that the loading places pointed toward the cathode and the gel lay horizontally and plane-parallel to the voltage used throughout the electrophoresis. Care was taken that the temperature was not below 8° C. and not above 14° C. during the electrophoresis.

The sample loading took place after the preliminary electrophoresis. The samples were boiled in sample buffer (preferably, for example, SDS- and bromophenol blue-containing sample buffer from Anamed or Novex) in a waterbath for 3 minutes and loaded in 20 μl portions per lane in the gel wells.

The sample electrophoresis took place at 25 V/max. watt/0.5 h. It caused penetration of the samples into the gel matrix.

The main electrophoresis took place (immediately following the sample electrophoresis) at 50 V/max. power, until the bromophenol blue front had reached the end of the gel.

For the Coomassie blue staining (SAGE-CB), the gels (preferably agarose gels on sheet) were fixed for example in a mixture of ethanol, acetic acid and water (25/3/72; v/v) for 0.5 h. The gels were then squeezed under pressure (for example by putting on a filled 10 l laboratory bottle) with a layer of filter paper until the agarose appeared dull (about 2-3 h) and then dried further in a drying oven at 40° C. until the agarose appeared transparent (about 1-2 h). The gels were subsequently stained with Coomassie blue with gentle shaking at 40° C. overnight (0.01% Serva blue G, 6% ammonium nitrate, 0.2% sodium acetate, 4 ml/l 65% HNO₃). The next morning, the gels were rinsed once briefly with water. Dye adhering to the gel support was removed by pulling off the backing side over a filter paper soaked with alcohol. The gels were then again squeezed under pressure with a layer of filter paper until the agarose appeared dull (about 1 h) and then again dried further in a drying oven at 40° C. until the agarose appeared transparent (about 1-2 h).

Finally, the dried gel was sealed or laminated in a special film in order to preserve it for evaluation and long-term documentation and archiving.

For the Western blot immunostaining (SAGE-WISA), for which agarose gels without backing were preferably employed, the blot buffer was made up freshly on the morning of the day of analysis (35.6 g Na₂HPO₄×2H₂O, 6.9 g NaH₂PO₄×H₂O and 10 ml of a 20% strength aqueous SDS solution were made up to 5,000 ml with demineralized water), cooled to +12° C. to +14° C. in a refrigerator (duration about 3 h) and then introduced into the blot chamber and the reaction dishes. The blot chamber cooling was adjusted to +8° C. The membrane (a nitrocellulose or— preferably—PVDF membrane, preferably an Immobilon-P 0.45 μm transfer membrane from Millipore) and a piece of paperboard (for example 165×65, Sarstedt AG) cut to 100×100 mm were equilibrated before the blotting in blot buffer while shaking gently for at least 0.5 h.

The complete blot sandwich was assembled in the blot buffer dish filled with blot buffer as follows:

1. blot frame (minus frame)

2. thin sponge sheet

3. paperboard (100×100 mm)

4. blot protecting membrane

5. agarose gel (preferably two filter strips (100×10 mm) were placed in each case to the left and right of the gel), then the bubbles were removed

6. blot membrane (0.45 μm)

7. paperboard (100×100 mm)

8. blot frame (plus frame)

Air bubbles under the gel were removed with the fingers (protective gloves).

The blotting took place by methods known per se to the skilled worker while cooling to 8±2° C. and stirring at 20 V plus max. power for a period of 75 min.

The blot sandwich was then disassembled and the blot membrane was placed in a reaction dish with blocking solution (the side which lay on the gel must be on top). The blot membrane was blocked in BSA buffer (1% bovine serum albumin (BSA) in tris-buffered saline, pH 8.0) while shaking for 15 min. The immunostaining was then carried out.

1. Incubation with first antibody (50 μl of rabbit anti-vWF (for example rabbit/anti-human/humain, DAKO) in 0.5% BSA buffer) for 45 min.

2. Rinse membrane three times briefly with demineralized water.

3. Wash with 50 ml of tris-buffered saline with 0.5% BSA buffer for 15 min

4. Incubation with second antibody (for example 50 μl of goat anti-rabbit IgG-AP (alkaline phosphatase-labeled; Sigma) in 50 ml of 0.5% BSA buffer) for 45 min.

5. Rinse membrane three times briefly with demineralized water.

6. Wash with 50 ml of BSA buffer for 15 min.

7. Incubate membrane in tub with 50 ml of substrate solution (2 tablets of BCIP/NPT (Sigma) ad 50 ml 0.5% BSA buffer) at room temperature for 15 min

8. Stop the reaction by washing several times with demineralized water

9. Place blotting membrane on a hotplate which has been heated to +40° C., cover with a piece of paperboard and remove the excess moisture by using a roller

10. Finally, the blot membrane was sealed or laminated in a special film in order to preserve it for evaluation and long-term documentation and archiving.

Documentation of the blot or of the gel stained with Coomassie blue took place as original in laminated form or by photographing or scanning.

The stained gel (laminated) or the immunostained blot membrane (laminated) was passed to a scanner and evaluated by densitometry using suitable software (for example using the “1-D-Elite”, Version Image Master 4.10 software from Pharmacia) and assessed in the usual way, for example as follows:

The contents of bands 1-5, 6-10 and 11 and higher were totaled by the evaluation software and related as percentage content in each case to the complete lane which was used as 100% basis.

The content of bands 11 and higher of each sample was related as percentage content to bands 11 and higher of the standard human plasma (=100%).

B. Determination of Fibrinogen Multimers

The agarose gel employed for separating the fibrinogen multimers had an agarose concentration of from 1.6 to 3.0 percent by weight, preferably from 1.8 to 2.4 percent by weight. An agarose gel without backing (e.g. from Bio-Rad) proved to be particularly advantageous for subsequent Western blot immunostain analysis (WISA). An agarose gel which proved to be particularly advantageous for Coomassie blue staining was one applied to a backing sheet (e.g. from Elchrom Scientific AG) because this allowed the agarose to be dried better at elevated temperature, preferably between 30° C. and 50° C., and more rapidly stained with Coomassie blue.

The submarine electrophoresis took place with cooling to 10° C. in an SEA 2,000 HIPER electrophoresis chamber (Elchrom Scientific, Cham, Switzerland) with 1.6-3% by weight, preferably 1.8 to 2.4% by weight agarose gels and, for example, an electrophoresis buffer composed of tris-glycine buffer with 0.1% SDS (mixture: 12.12 g of tris, 57.8 g of glycine and 2 g of SDS were made up to 2,000 ml with deionized water). The electrophoresis cell was charged with 2,000 ml of electrophoresis buffer. The gel was then inserted and re-equilibrated in a preliminary electrophoresis. The preliminary electrophoresis took place at 25 V/max. watt/0.5 h (with circulating pump running in order to make rapid re-equilibration of the gel possible).

For the electrophoresis, the gel was placed in the electrophoresis chamber in such a way that the loading places pointed toward the cathode and the gel lay horizontally and plane-parallel to the voltage used throughout the electrophoresis. Care was taken that the temperature was not below 8° C. and not above 14° C. during the electrophoresis.

The sample loading took place after the preliminary electrophoresis. The samples were boiled in sample buffer (preferably, for example, SDS- and bromophenol blue-containing sample buffer from Anamed or Novex) in a waterbath for 3 minutes and loaded in 20 μl portions per lane in the gel wells.

The sample electrophoresis took place at 25 V/max. watt/0.5 h. It caused penetration of the samples into the gel matrix.

The main electrophoresis took place (immediately following the sample electrophoresis) at 50 V/max. power/0.5 h; then 75 V/max. watt, until the bromophenol blue front had reached the end of the gel.

For the Coomassie blue staining, the gels (preferably agarose gels on sheet for example from Elchrom Scientific) were fixed for example in a mixture of ethanol, acetic acid and water (25/3/72; v/v) for 0.5 h. The gels were then squeezed under pressure with a layer of filter paper until the agarose appeared dull (about 2-3 h). Then the gels were dried further in a drying oven at 40° C. until the agarose appeared transparent (about 1-2 h). The gels were subsequently stained with Coomassie blue with gentle shaking at 40° C. overnight (0.01% Serva blue G, 6% ammonium nitrate, 0.2% sodium acetate, 4 ml/l 65% HNO₃). The next morning, the gels were rinsed once briefly with water. Dye adhering to the gel support was removed by pulling off the backing side over a filter paper soaked with alcohol. The gels were then again squeezed under pressure with a layer of filter paper until the agarose appeared dull (about 1 h) and then again dried further in a drying oven at 40° C. until the agarose appeared transparent (about 1-2 h).

Finally, the dried gel was sealed or laminated in a special film in order to preserve it for evaluation and long-term documentation and archiving.

For the Western blot immunostaining (SAGE-WISA), for which agarose gels without backing were preferably employed, the blot buffer was made up freshly on the morning of the day of analysis (35.6 g Na₂HPO₄×2H₂O, 6.9 g NaH₂PO₄×H₂O and 10 ml of a 20% strength aqueous SDS solution were made up to 5,000 ml with demineralized water), cooled to +12° C. to +14° C. in a refrigerator (duration about 3 h) and then introduced into the blot chamber and the reaction dishes. The blot chamber cooling was adjusted to +8° C. The membrane (a nitrocellulose or—preferably—PVDF membrane, preferably an Immobilon-P 0.45 μm transfer membrane from Millipore) and a piece of paperboard (for example 165×65, Sarstedt AG) cut to 100×100 mm were equilibrated before the blotting in blot buffer while shaking gently for at least 0.5 h.

The complete blot sandwich was assembled in the blot buffer dish filled with blot buffer as follows:

1. blot frame (minus frame)

2. thin sponge sheet

3. paperboard (100×100 mm)

4. blot protecting membrane

5. agarose gel (preferably two filter strips (100×10 mm) were placed in each case to the left and right of the gel), then the bubbles were removed

6. blot membrane (0.45 μm)

7. paperboard (100×100 mm)

8. blot frame (plus frame)

Air bubbles under the gel were removed with the fingers (protective gloves).

The blotting took place by methods known per se to the skilled worker while cooling to 8±2° C. and stirring at 20 V at max. power for a period of 75 min.

The blot sandwich was then disassembled and the blot membrane was placed in a reaction dish with blocking solution (the side which lay on the gel must be on top). The membrane was blocked in BSA buffer (1% bovine serum albumin (BSA) in tris-buffered saline, pH 8.0) while shaking for 15 min. The immunostaining was then carried out.

1. Incubation with first antibody (50 μl of rabbit anti-fibrinogen (for example rabbit/anti-human/humain, DAKO) in 0.5% BSA buffer) for 45 min.

2. Rinse membrane three times briefly with demineralized water.

3. Wash with 50 ml of tris-buffered saline with 0.5% BSA buffer for 15 min

4. Incubation with second antibody (for example 50 μl of goat anti-rabbit IgG-AP (alkaline phosphatase-labeled; Sigma) in 50 ml of 0.5% BSA buffer) for 45 min.

5 Rinse membrane three times briefly with demineralized water.

6. Wash with 50 ml of BSA buffer for 15 min.

7. Incubate membrane in tub with 50 ml of substrate solution (2 tablets of BCIP/NPT (Sigma) ad 50 ml 0.5% BSA buffer) at room temperature for 15 min

8. Stop the reaction by washing several times with demineralized water

9. Place blotting membrane on a hotplate which has been heated to +40° C., cover with a piece of paperboard and remove the excess moisture by using a roller

10. Finally, the blot membrane was sealed or laminated in a special film in order to preserve it for evaluation and long-term documentation and archiving.

Documentation of the blot or of the gel stained with Coomassie blue took place as original in laminated form or by photographing or scanning.

The stained gel (laminated) or the immunostained blot membrane (laminated) was passed to a scanner and evaluated by densitometry using suitable software (for example using the “1-D-Elite”, Version Image Master 4.10 software from Pharmacia).

C. Evaluation of the Fractionation of the vWF Multimers

### EXAMPLE 1

SAGE-WISA of serial dilutions of standard human plasma in a bio-Rad precast 1% agarose gel.

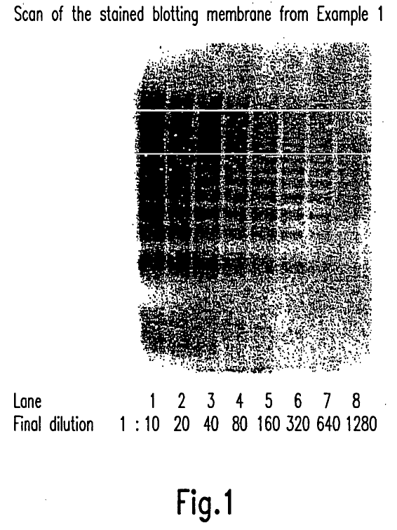

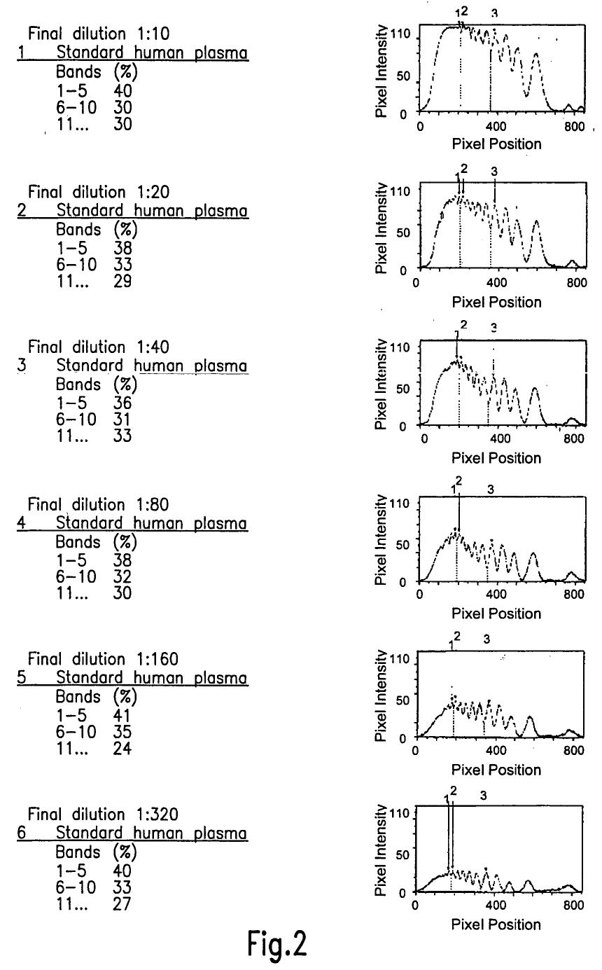

Standard human plasma (Dade Behring) was diluted with demineralized water in serial geometric dilutions. The dilutions were mixed 1:1 (v/v) with sample buffer so that the final concentrations indicated in FIG. 1 resulted. The samples were boiled in sample buffer (preferably, for example SDS- and bromophenol blue-containing sample buffer from Anamed or Novex) in a waterbath for 3 minutes and loaded in 20 μl portions per lane in the gel wells. The scan of the stained blotting membrane is shown in FIG. 1 and its densitometric evaluation in FIG. 2. The peak groups were quantified in analogy to (4).

### EXAMPLE 2

SAGE-WISA of serial dilutions of Haemate-HS® in a Bio-Rad precast 1% agarose gel.

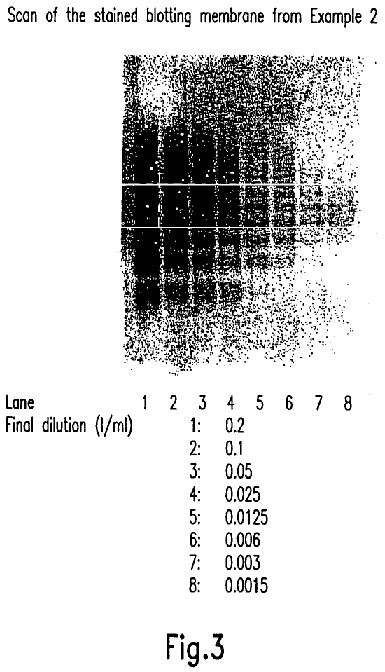

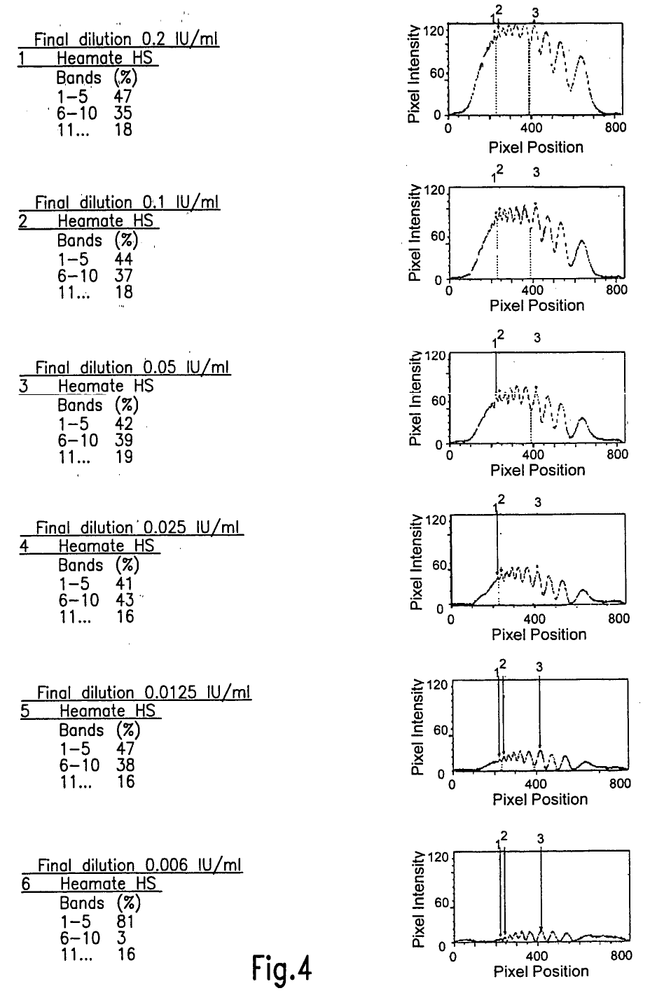

Haemate-HS® (Aventis Behring) was diluted with demineralized water in serial geometric dilutions. The dilutions were mixed 1:1 (v/v) with sample buffer so that the final concentrations indicated in FIG. 3 resulted. The samples were boiled in a waterbath for 3 minutes and loaded in 20 μl portions per lane in the gel wells. The scan of the stained blotting membrane is shown in FIG. 3 and its densitometric evaluation in FIG. 4. The peak groups were quantified in analogy to (4).

### EXAMPLE 3

SAGE-CB of Humate-P® and Haemate-HS® in an Elchrom Scientific AG precast 1% agarose gel

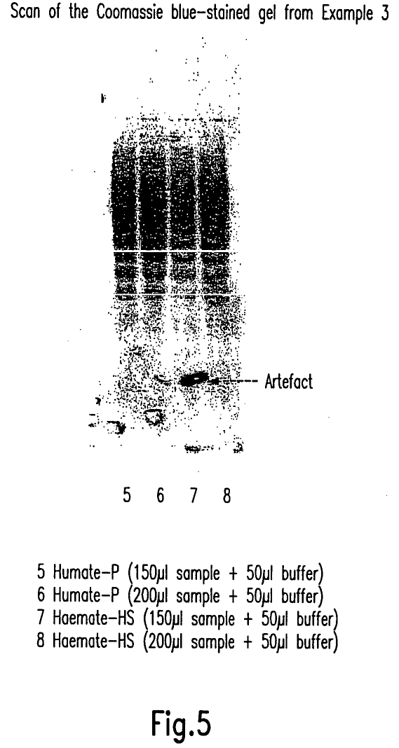

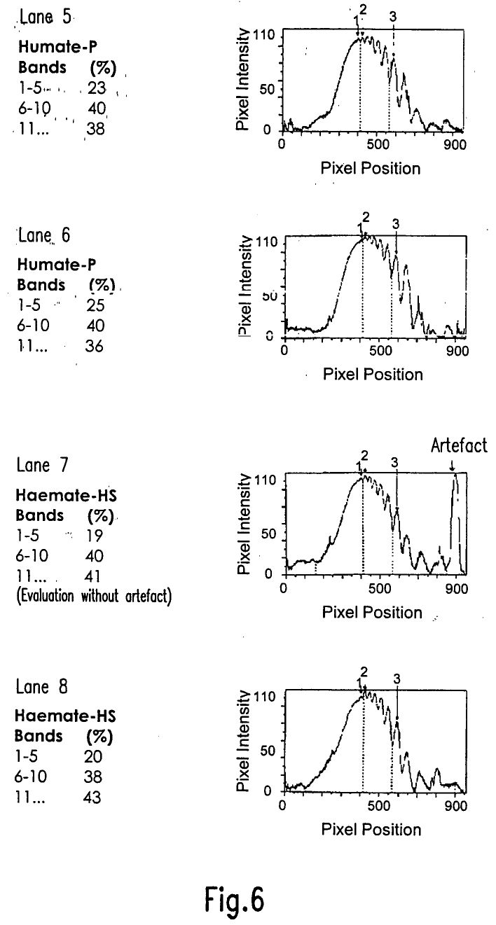

Humate-P® and Haemate-HS® were diluted with sample buffer to the concentrations shown in FIG. 5, boiled in a waterbath for 3 min and loaded in 20 μl portions per lane in the gel wells. The scan of the stained gel is shown in FIG. 5, and its densitometric evaluation in FIG. 6. The peak groups were quantified in analogy to (4).

Comparison of the densitometric evaluations of the vWF samples from FIG. 4 (blot membrane) and FIG. 6 (blue staining in the gel) shows the percentage content of bands 11 and higher in the Western blot is about 18%, but is virtually twice as high in the blue staining, at about 40%. This finding underlines the observation disclosed in (8) that the high molecular weight bands are blotted less well. Perutelli has overcome this weakness of the Western blot by a direct staining of, the multimer bands in the gel using a radioactive antibody conjugate (autoradiography or luminography). By contrast, the blue stain used according to the invention has the advantage that it is completely non-hazardous, although less sensitive.

D. Evaluation of the Fractionation of the Fibrinogen Multimers

### EXAMPLE 4

SAGE-CB of fibrinogen in an Elchrom Scientific AG precast 2% agarose gel.

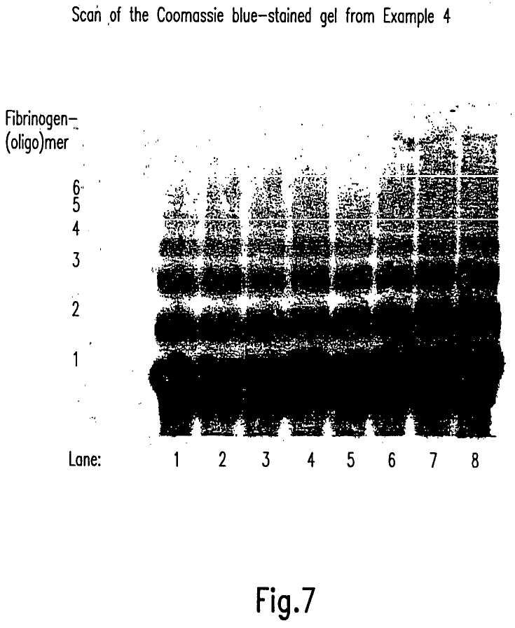

The samples were mixed 1:1 (v/v) with sample buffer, boiled in a waterbath for 3 min and loaded in 20 μl portions (≈360 μg fibrinogen; 18 mg/ml) per lane in the gel wells. The scan of the Coomassie blue-stained gel is shown in FIG. 7.

### EXAMPLE 5

SAGE-WISA of fibrinogen in an Elchrom Scientific precast 2% agarose gel.

The fibrinogen samples (90 g/l) were diluted 1:100 (v/v) with demineralized water and then 1:12 (v/v) with sample buffer, boiled in a waterbath for 3 min and loaded in 20 μl portions per lane into the gel wells.

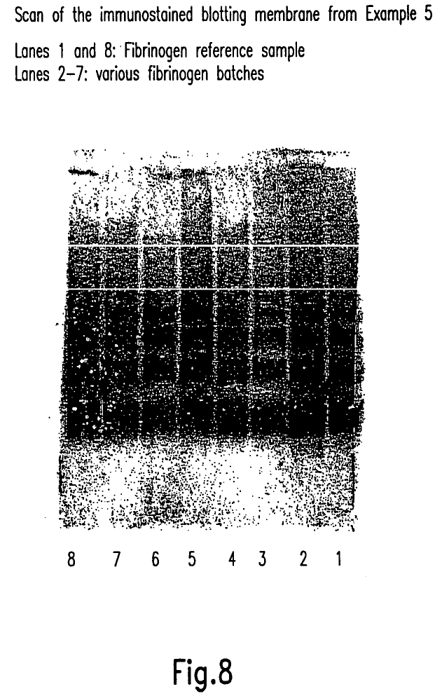

The scan of the immunostained blotting membrane is shown in FIG. 8. In this case, the agarose gel was detached from the backing sheet for the blotting.

### EXAMPLE 6

SAGE-WISA of Humate-P® in an Elchrom Scientific precast 2% agarose gel.

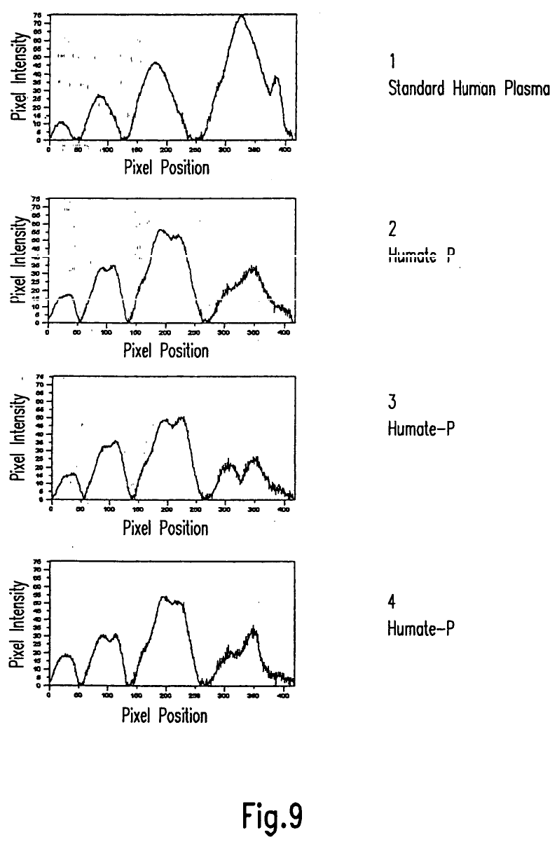

The Humate sample was prediluted with MQ water in analogy to Example 2 and mixed 1:1 with sample buffer so that the final concentration indicated in FIG. 3, lane 2, resulted (0.1 IU/ml). The Humate sample was boiled in a waterbath for 3 min and measured in 20 μl portions per lane in gel wells 2 to 7 versus standard human plasma (lane 1 and lane 8) in a 2% agarose gel from Elchrom Scientific, with the main electrophoresis being increased from 3 to 4.5 hours compared with the measurement in the 1% gel. Blotting and gel staining took place in analogy to Example 2. The scan of the stained blotting membrane and densitometric evaluation of the low molecular weight region of lanes 1 to 4 is shown in FIG. 9. In this case, the agarose gel was detached from the backing sheet for the blotting. Compared with standard human plasma, the low molecular weight multimer bands of Humate-P showed a fine structure (split into double bands).

List of References:

1. Connaghan, D. G., Francis, C. W., Lane, D. A.; Marder, V. J. (1985) Specific identification of fibrin polymers, fibrinogen degradation products and crosslinked fibrin degradation products in plasma and serum with a new sensitive technique. Blood 65 (3) 589-97.

2. Proietti, A. B.; McGuire, M.; Bell, W. (1990) Specific identification of fibrin(ogen) degradation products in plasma and serum using blotting and peroxidase labeled antiserum. American Journal of Hematology 34 (4) 270-4.

3. Raines, G.; Aumann, H.; Sykes, S.; Street A. (1990) Multimeric analysis of von Willebrand factor by molecular sieving electrophoresis in sodium dodecyl sulphate agarose gel. Thrombosis Research 60 (3) 201-12.

4. Metzner, H. J.; Hermentin, P.; Cuesta-Linker, T.; Langner, S.; Müller, H.-G. and Friedebold, J. (1998) “Characterization of factor VIII/von Willebrand factor concentrates using a modified method of von Willebrand factor multimer analysis” Haemophilia (1998), 4 (Suppl. 3), 25-32.

5. Coon and Bayer (1995) Clin. Appl. Thrombosis/Hemostasis 1, 31-3

6. Tatewaki et al., (1989) Thromb. Res. 56, 191-9

7. Ruggeri and Zimmermann (1981) Blood 57: 1140-3

8. Perutelli, P., Haematologica 2002; 87:223-224

1. A method for the qualitative and quantitative determination of the multimers of multimer-forming therapeutic proteins by gel electrophoresis, wherein a sample containing von Willebrand factor (vWF) or fibrinogen is fractionated by submarine electrophoresis using a continuous, homogeneous agarose gel free of lumps, and the multimer bands are visualized immunochemically after a Western blot analysis by a specific antibody-enzyme conjugate on the blotting membrane or by a suitable dye, preferably with a blue stain, in the gel.

2. The method as claimed in claim 1, wherein the multimer-forming therapeutic protein is fibrinogen.

3. The method as claimed in claim 1, wherein the multimer-forming therapeutic protein is von Willebrand factor (vWF).

4. The method as claimed in claim 2, wherein an agarose gel with an agarose concentration of 1.6-3% by weight, preferably of 1.8-2.4% by weight, is employed for separating the fibrinogen multimer.

5. The method as claimed in claim 3, wherein an agarose gel with an agarose concentration of 0.7-1.8% by weight, preferably of 0.8-1.2% by weight is employed for separating the vWF multimers.

6. The method as claimed in claims 1 to 5, wherein the gel electrophoresis is carried out at temperatures between 6° C. and 14° C., preferably between 8° C. and 12° C.

7. The method as claimed in claims 1 to 6, wherein an immunostaining is employed for staining the multimer bands on the blotting membrane.

8. The method as claimed in claims 1 to 6, wherein the dye Coomassie blue is employed for blue staining of the multimer bands in the gel.

9. The method as claimed in claims 1 to 8, wherein an agarose gel on a backing sheet is employed for the blue staining in the gel.

10. The method as claimed in claims 1 to 8, wherein an agarose gel without backing sheet is employed for the immunostaining on the blotting membrane, or the backing sheet is removed from the gel before the blotting process.

11. The method as claimed in claims 1 to 10, wherein the bands are quantified by densitometry.

12. The method as claimed in claim 11, wherein the bands are quantified after blue staining of the gel.

13. The method as claimed in claim 11, wherein the bands are quantified after immunostaining of the blotting membrane.

14. The method as claimed in claim 9, wherein the gel is preserved by lamination after the staining.

15. The method as claimed in claim 10, wherein the blotting membrane is preserved by lamination after the immunostaining.
